(19) United States
(12) Patent Application Publication
(10) Pub. No.: US 2026/0346160 A1
(43) Pub. Date: Nov. 5, 2026

(54) INTEGRATION OF A HEAT PUMP CIRCUIT IN A DISTILLATION PLANT FOR POLYMERISABLE SUBSTANCES

(71) Applicant: BASF SE, Ludwigshafen am Rhein (DE)

(72) Inventors: Jonathan WALTER, Ludwigshafen am Rhein (DE); Marvin KRAMP, Ludwigshafen am Rhein (DE); Ortmund LANG, Ludwigshafen am Rhein (DE); Cornelis Hendricus DE RUITER, Ludwigshafen am Rhein (DE)

(21) Appl. No.: 19/101,912

(22) PCT Filed: Aug. 1, 2023
(86) PCT No.: PCT/EP2023/071235

§ 371 Date: Feb. 7, 2025

(30) Foreign Application Priority Data
Aug. 9, 2022 (EP) 22189502.2

(51) Int. Cl. B01D 3/00; B01D 3/32; C07C 67/08; C07C 67/54; C07C 69/54

(52) U.S. Cl. CPC B01D 3/007; B01D 3/322; C07C 67/08; C07C 67/54; C07C 69/54

(57) ABSTRACT

The invention relates to a distillation plant for polymerizable substances comprising a rectification column (1) having a reboiler (2) and a vapor condenser (3), wherein a heat pump circuit comprising a compressor (7) fluidically connects both the reboiler (2) and the vapor condenser (3) and is configured such that during operation of the distillation plant the reboiler (2) is heated by the heat pump circuit and the vapor condenser (3) is cooled by the heat pump circuit, wherein the heat pump circuit employs water as the working medium, wherein a mixing element (8) is arranged between the compressor (7) and the downstream reboiler (2), wherein the mixing element (8) is configured such that during operation of the distillation plant in order to cool the working medium a cooling water is added in an amount such that a temperature of the working medium in the range from 80 °C to 200 °C can be established at the inlet of the reboiler (2) and wherein the distillation plant is configured such that during operation of the distillation plant the working medium partially serves as cooling water, wherein the working medium serving as cooling water is withdrawn from a subregion of the heat pump circuit which extends in the main flow direction of the working medium from the reboiler (2) to the vapor condenser (3).

The invention further relates to a process for producing a (meth)acrylate, to the use of a distillation plant and to a method of operating a distillation plant.

The present invention relates to a distillation plant for polymerizable substances comprising a rectification column having a reboiler and a vapor condenser, wherein a heat pump circuit comprising a compressor fluidically connects both the reboiler and the vapor condenser and is configured such that during operation of the distillation plant the reboiler is heated by the heat pump circuit and the vapor condenser is cooled by the heat pump circuit, wherein the heat pump circuit employs water as the working medium.

Distillation plants for polymerizable substances, in particular (meth)acrylates, generally have the problem that during operation of the distillation plant excessively high temperatures of the polymerizable substances can lead to severe polymerization within the distillation plant. The reboilers of the distillation plants are often affected by severe polymerization and/or by significant fouling because of their high heating temperature. Accordingly, considerable deposits can form on the interior walls of the flow channels of the reboiler. The flow channels of the polymerizable substances may therefore block in operation of the distillation plant with the result that the reboiler loses its ability to function.

Due to the energy-intensive method of operating such a distillation plant a heat pump is generally an option for making the energy content of the vapor stream at least partially reusable for the distillation plant.

JPS 60125201 A discloses a distillation plant for polymerizable substances, where a heat pump circuit comprising a compressor fluidically connects both a reboiler and a vapor condenser. The reboiler is heated by the heat pump circuit and the vapor condenser is cooled by the heat pump circuit. Water is employed as the working medium of the heat pump circuit. However, this disclosure has the disadvantage that compression of the working medium by the compressor can also cause the temperature of the working medium to increase excessively, with the result that the downstream reboiler becomes too hot for the polymerizable substances flowing through it.

EP 0965373 A1 discloses a distillation plant for polymerizable substances comprising a substitutable heat pump. The distillation plant comprises a column and a heat pump operating between a reboiler and a vapor condenser of the column. The heat pump is substitutable by means which, when required, are connectable to the reboiler and the vapor condenser. These substituting means comprise means for producing steam/for producing a coolant. However, when the heat pump is operated this implementation too has the disadvantage that compression of the working medium by the compressor can also cause the temperature of the working medium to increase excessively, with the result that the downstream reboiler becomes too hot for the polymerizable substances flowing through it.

It is accordingly an object of the present invention to ensure that the allowable temperature range for the polymerizable substances in the reboiler is maintained during operation of the distillation plant. It is a further object of the invention to be able to adjust within the distillation plant the optimal temperature of the working medium in the reboiler for the prevailing operating conditions in a manner that is faster, more stable and more precise.

These objects are achieved in accordance with the present invention by a distillation plant according to claim 1, by a process for producing a (meth)acrylate according to claim 9, by the use of a distillation plant according to claim 15 and by a method of operating the distillation plant according to claim 16. The invention further relates to preferred embodiments of the distillation plant according to claims 2 to 8 and to preferred embodiments of the process for producing a (meth)acrylate according to claims 10 to 14.

According to the invention the distillation plant for polymerizable substances comprises a rectification column having a reboiler and a vapor condenser, wherein a heat pump circuit comprising a compressor fluidically connects both the reboiler and the vapor condenser and is configured such that during operation of the distillation plant the reboiler is heated by the heat pump circuit and the vapor condenser is cooled by the heat pump circuit, wherein the heat pump circuit employs water as the working medium, wherein a mixing element is arranged between the compressor and the downstream reboiler, wherein the mixing element is configured such that during operation of the distillation plant a cooling water is added to cool the working medium.

The cooling water is added to cool the working medium in an amount such that a temperature of the working medium in the range from 80 °C to 200 °C, preferably from 100 °C to 160 °C, in particular from 110 °C to 160 °C, can be established or is established or is present at the inlet of the reboiler.

The addition of a cooling water to the working medium between the compressor and the downstream reboiler provides the advantage that the temperature of the working medium at the inlet of the reboiler may be controlled in a manner that is fast, stable and precise. This makes it possible to avoid elevated temperature peaks before they reach the reboiler through the addition of the cooling water. The allowable temperature range for the polymerizable substances in the reboiler is thus maintained during operation of the distillation plant.

In the present document the term “rectification column” is to be understood as meaning a general description for apparatuses which, through heating, produce vapors which ascend and are in contact with a descending liquid phase.

Rectification columns are known in their general construction and comprise the customary apparatuses such as for example an evaporator in the column bottom, an evaporator in the high-boilers outflow or a condenser in the low-boilers outflow, wherein the high-boilers are preferably in the bottom region and the low boilers are preferably in the top region of the rectification column. Typically a portion of the mass flow of the high-boilers outflow is recycled into the bottom region of the rectification column. However, it is in principle also possible for the bottom region to be heated via for example an outer wall heating of the column in the bottom region and/or for an evaporator to be integrated in the bottom region. Typically a vapor stream is withdrawn at the top of the rectification column and supplied to a condenser. The vapor stream is typically also referred to as low-boilers outflow. A portion of the vapor stream condensed in the condenser is recycled into the rectification column while the remaining portion of the condensed vapor stream is discharged as distillate. The reflux ratio describes the ratio between the condensed vapor stream recycled into the column and the condensed vapor stream withdrawn as distillate. A reflux ratio in the range from 10% to 200% is generally established. Contemplated column internals for the rectification column in principle include all commonly used internals, for example trays, structured packings and/or random packings. Preferred trays include bubble cap trays, sieve trays, valve trays, Thormann trays and/or dual-flow trays, while preferred random packings include those comprising rings, coils, saddles or weaves. The rectification column may also comprise further standard components for control, for example pressure regulators, flow controllers or sensors. It is in principle also possible to connect two or more rectification columns to one another in series or parallel which can then also act as a single “rectification column” in their integrated system.

In the present document the term “reaction zone” is to be understood as meaning that a chemical reaction may occur within one zone, wherein the reaction zone may be in a reactor, in the bottom of a rectification column or in a reactive distillation column for example. In the preferred case where the reaction zone is in a reactor the reactor may comprise a column placed atop it, wherein in the case of esterification processes occurring in the reactor the column preferably effects distillative separation of water. The column is itself generally a distillation column or a rectification column comprising internals. Such internals are trays, such as bubble cap trays, perforated trays, in particular dual-flow trays, random packings, structured packings or the like. The reactor may also be integrated into the rectification column so that the reaction can occur in the bottom of the rectification column.

In the present document the term “reboiler” is to be understood as meaning a general heating element for a rectification column. The reboiler typically heats a bottoms mixture from the rectification column in such a way that a bottoms outflow flows through the reboiler and is subsequently returned to the bottom of the rectification column. The reboiler may also comprise further standard components such as for example control valves, pressure regulators, flow controllers or sensors. The evaporator may thus also comprise a control means. The term “reboiler” can generally also be understood as meaning two or more evaporators connected to one another in series or parallel. Examples of suitable reboilers include thin film evaporators, Robert evaporators, falling film evaporators, natural circulation and forced circulation evaporators. The evaporators may be in the form of shell-and-tube heat exchangers or plate heat exchangers. A person skilled in the art is familiar with suitable evaporators and these are described for example in SPX, Evaporator Handbook, APV Americas, Engineered Systems, Separation Technologies, 4th Edition, available at https://userpages.umbc.edu/~dfrey1/ench445/apv_evap.pdf (retrieved on 20.05.2022). In the heat pump circuit the reboiler functions as a condenser which at least partially, preferably completely, condenses the working medium via the bottoms outflow.

In the present document the term “water separator” is to be understood as meaning an apparatus which can separate water droplets from a gas stream for example. Typical examples of a water separator include a demister, in particular a demister comprising knitted wire mesh, a centrifugal droplet separator or a lamellar separator. The water separator may be arranged in a water collection vessel or be configured as a dedicated apparatus.

In the present document the term “vapor condenser” is to be understood as meaning an apparatus which cools and condenses a vapor stream from a distillation plant. Typical examples of a vapor condenser include a shell-and-tube heat exchanger, a jacketed-tube heat exchanger or a plate heat exchanger. The heat exchanger may be provided with nozzles for injecting a solution comprising polymerization inhibitor(s) to prevent polymer formation. In the heat pump circuit the vapor condenser functions as an evaporator which at least partially, preferably completely, evaporates the working medium via the heat-supplying vapor stream.

In the present document the term “mixing element” is to be understood as meaning an apparatus which admixes a liquid stream with a further liquid stream. The mixing element is in particular a component separate from the compressor. Furthermore, the mixing element is preferably arranged downstream of the compressor. Typical examples of a mixing element include a steam jet, a venturi mixer, one or more mixing nozzles or a long pipe serving as a mixing path. In the case of a pipe of several meters long, mixing nozzles are arranged individually in the pipe. Alternatively, the mixing nozzles are provided by a coaxial pipe in the form of openings in the intermediate wall.

In the present document the term “compressor” is to be understood as meaning a machine which compresses gases. One example of a suitable compressor is a geared turbocompressor. This is generally provided with two or more compression stages and intermediate stages, wherein the respective intermediate stage is provided with an apparatus for intermediate cooling.

In the present document the term “working medium” is generally to be understood as meaning the fluid which flows through the heat pump circuit and especially through its local phase changes can supply or remove heat.

In the present document a “heat pump” is generally to be understood as meaning a machine which by doing technical work absorbs thermal energy from a reservoir having a relatively low temperature and, together with the propulsion energy, transfers this as useful heat to a system to be heated having a relatively high temperature. The system to be heated may be a reboiler for example and the reservoir having a relatively low temperature may be a vapor condenser for example.

In the present document the term “heat pump circuit” is generally to be understood as meaning a circuit through which a working medium circulates. The heat pump circuit includes a compressor, a condenser and an evaporator, each of which is fluid-tightly connected to the heat pump circuit. In the heat pump circuit the reboiler functions as the condenser in the heat pump circuit and the vapor condenser functions as the evaporator in the heat pump circuit.

In the present document the term “cooling water” is to be understood as meaning a water which is used for cooling the working medium in the compressor or for cooling the working medium at a point between the compressor and the downstream reboiler in the heat pump circuit. Cooling water may generally be supplied to the working medium from outside the heat pump circuit via a conduit into the heat pump circuit. According to the invention, the cooling water is provided via a diversion of a substream of the working medium at a point in the heat pump circuit between the reboiler and a downstream vapor condenser.

In the present document the term “fluidically connect” is to be understood as meaning that generally two or more flow-conducting components such as for example two or more flow tubes are connected to one another in such a way that these connected components are able to conduct a flow of a fluid. A sufficient technical fluid-tightness should generally be ensured upon conduction of a flow of fluid.

In a preferred embodiment of the distillation plant the mixing element is configured such that during operation of the distillation plant in order to cool the working medium a cooling water having a temperature in the range from 1 °C to 160 °C, more preferably from 105 °C to 150 °C, in particular from 110 °C to 140 °C, is added to the working medium, wherein the cooling water has a mass flow rate relative to the mass flow rate of the working medium in the range from 3% to 10%. This has the advantage that the optimal temperature of the working medium at the inlet of the reboiler may be adjusted in a manner that is fast, stable and precise.

The distillation plant is configured such that during operation of the distillation plant the working medium partially serves as cooling water, wherein the working medium serving as cooling water is withdrawn from a subregion of the heat pump circuit which extends in the main flow direction of the working medium from the reboiler to the vapor condenser. In particular, the distillation plant has a return conduit which fluidically connects the outlet of the reboiler in the heat pump circuit to the mixing element. The return conduit is in particular arranged at the subregion of the heat pump circuit which extends in the main flow direction of the working medium from the reboiler to the vapor condenser. The working medium serving as cooling water is preferably withdrawn and/or returned at an absolute pressure in a range from 2 to 8 bar, more preferably from 4 to 6 bar. The working medium serving as cooling water is preferably withdrawn and/or returned at a temperature in a range from 105 °C to 150 °C, more preferably from 110 °C to 140 °C.

It has surprisingly been found that the working medium cooled by the reboiler may be partially used as cooling water. This technical implementation has the advantage that no further external cooling water is required and the heat pump circuit is not disrupted by external cooling water except in exceptional cases. An example of an exceptional case would be if the working medium drains from the heat pump circuit as a result of a leak or if the working medium requires a partial replacement by fresh working medium. Complex safety apparatuses such as pressure valves or complex pressure-attenuating means become redundant. In addition the efficiency of the heat pump is increased compared to cooling with a cooling medium outside the heat pump circuit.

In a preferred embodiment of the distillation plant the vapor condenser is an upright shell-and-tube heat exchanger. Space can therefore be saved in the distillation plant and injection of a solution comprising polymerization inhibitor(s) is technically easily realizable.

In a preferred embodiment of the distillation plant the reboiler has a downstream condensate vessel in the heat pump circuit and both the condensate vessel and the mixing elements are adapted such that during operation of the distillation plant the working medium partially flows from the condensate vessel to the mixing element while serving as cooling water. If the working medium is collected in a condensate vessel downstream of the reboiler this has the advantage that sufficient working medium serving as cooling water is present in the case of a reduction in the mass flow rate of the working medium.

In a preferred embodiment of the distillation plant both the condensate vessel and the compressor are adapted such that during operation of the distillation plant the working medium partially flows from the condensate vessel to the compressor while serving as cooling water. This has the advantage that this also cools the compressor without the need to employ other fluids. The condensate vessel allows intermediate storage of the working medium with the result that sufficient working medium serving as cooling water is always present between the compressor and the downstream reboiler both for the cooling of the compressor and for the cooling of the working medium.

In a preferred embodiment of the distillation plant the compressor comprises one compression stage or two or more compression stages and the distillation plant is adapted such that during operation of the distillation plant the cooling water in the case of two or more compression stages is at least partially added to the working medium via a respective intermediate-stage mixing element per compression stage, wherein the respective intermediate-stage mixing element is arranged between the respectively adjacent compression stages of the compressor. The respective intermediate-stage mixing element is preferably fluidically connected via the return conduit to the outlet of the reboiler in the heat pump circuit. In the case of multiple compression stages this has the advantage that the working medium is cooled between the stages without having to employ other fluids.

In a preferred embodiment of the distillation plant a liquid droplet separator or a tube having a length-to-diameter ratio of at least 10 is connected downstream of the respective intermediate-stage mixing element. This protects the downstream compression stage from droplets that can damage the compressor.

In a preferred embodiment of the distillation plant the distillation plant is configured such that during operation of the distillation plant in order to cool the working medium a cooling water having a temperature in the range from 1 °C to 160 °C, more preferably from 105 °C to 150 °C, in particular from 110 °C to 140 °C, is added to the working medium via the intermediate-stage mixing element in an amount such that at the inlet of the downstream compression stage a temperature difference between the temperature of the working medium and the temperature at which under the prevailing absolute pressure the working medium is in the form of saturated steam in the range from 2 to 50 °C, preferably in the range from 5 to 20 °C, can be established. In this embodiment the cooling water is preferably added via the intermediate-stage mixing element at a mass flow rate relative to the mass flow rate of the working medium at the compressor inlet in the range from 3% to 10%. This has the advantage that the cooling water is energy-efficiently added to the working medium between the compression stages so that only as much cooling water as necessary is added.

In a preferred embodiment of the distillation plant the mixing element and/or the intermediate-stage mixing element is a mixing nozzle, a steam jet or a venturi pump. These variants bring about an energy-efficient, robust and economical industrial implementation.

In a preferred embodiment of the distillation plant the distillation plant is configured such that during operation of the distillation plant a further vapor condenser downstream of the vapor condenser is present and this further vapor condenser comprises a downstream phase separator, wherein the phase separator is configured such that during operation of the distillation plant both an aqueous phase and an organic phase can form, wherein one of these two phases is preferably at least partially recycled to the rectification column and wherein very particularly preferably both phases are each separately at least partially recycled to the rectification column through two separate recycle conduits. This has the advantage that the amount of aqueous phase can be recycled to the rectification column irrespective of the amount of organic phase, and vice versa.

In a preferred embodiment of the distillation plant the vapor condenser and the further vapor condenser are an upright shell-and-tube heat exchanger. An upright shell-and-tube heat exchanger has the advantage that a preferred addition of a solution comprising polymerization inhibitor(s) can be implemented more easily in the inlet region of the shell-and-tube heat exchanger.

In a preferred embodiment of the distillation plant a phase separator is connected downstream of the vapor condenser, wherein the phase separator is configured such that during operation of the distillation plant both an aqueous phase and an organic phase are formed in the phase separator and these two phases are at least partially recycled to the rectification column, wherein the two phases are preferably separately recycled to the rectification column via two separate recycle conduits. This has the advantage that in the case of processes having polymerizable substances optimal separation in the rectification column is achieved.

In a preferred embodiment of the distillation plant the distillation plant is configured such that during operation of the distillation plant the reboiler is configured such that the walls thereof, across which the bottoms mixture from the rectification column flows during operation of the distillation plant, are made from a nonrusting and acid-resistant material selected from the group consisting of zirconium, stainless steels and nickel-based alloys. This virtually or even completely prevents corrosion processes on the walls.

In a preferred embodiment of the distillation plant the heat pump circuit comprises a water separator, wherein the water separator is fluidically connected to the vapor condenser in such a way that during operation of the distillation plant the working medium flows from the water separator into the vapor condenser and then the working medium flows back from the vapor condenser into the water separator. The water separator serves as a collection vessel for the working medium, which means that, in the case of a reduction in the mass flow rate of the working medium, there will be both sufficient working medium to cool the vapor condenser and sufficient working medium for the compressor. In addition, the water separator attenuates possible pressure fluctuations in the heat pump circuit. There is preferably a demister in the water separator which protects the downstream compressor from damage by liquid droplets.

In a preferred embodiment of the distillation plant the distillation plant is configured such that during operation of the distillation plant in order to cool the working medium a cooling water having a temperature in the range from 1 °C to 160 °C, more preferably from 105 °C to 150 °C, in particular from 110 °C to 140 °C, is added to the working medium via the mixing element in an amount such that at the inlet of the reboiler a temperature difference between the temperature of the working medium and the temperature at which under the prevailing absolute pressure the working medium is in the form of saturated steam in the range from 5 °C to 50 °C, more preferably from 5 °C to 20 °C, can be established, wherein the prevailing absolute pressure is preferably in the range from 2 to 8 bar, particularly preferably in the range from 4 to 6 bar. In this embodiment the cooling water is preferably added via the mixing element at a mass flow rate relative to the mass flow rate of the working medium in the range from 3% to 10%. This has the advantage that the cooling water is energy-efficiently added to the working medium between the compressor and the downstream reboiler. Only as much cooling water as necessary is added so that the working medium transfers sufficient heat to the reboiler and the temperature of the working medium is not too high.

The invention further provides for the use of a distillation plant according to the invention, wherein the distillation plant is employed in chemical processes, in particular in processes for producing (meth)acrylates, preferably n-butyl (meth)acrylates, or in processes where (meth)acrylates, preferably n-butyl (meth)acrylates, occur. The use of a distillation plant according to the invention in such chemical processes offers the advantage that polymer formation in the heat pump circuit is avoided.

The invention further provides a method of operating the distillation plant.

According to the invention in the method of operating the distillation plant a liquid mixture is conveyed into the rectification column, wherein during the distillative separation a vapor stream having a temperature in the range from 35 °C to 120 °C, in particular from 50 °C to 100 °C, is formed at the inlet of the vapor condenser and a bottoms product having a temperature in the range from 80 °C to 160 °C, in particular from 80 °C to 130 °C, is formed in the bottom of the rectification column, and the working medium from the reboiler is at least partially supplied to the vapor condenser to be cooled, thus heating the working medium in the vapor condenser, wherein the working medium at the outlet of the vapor condenser has a temperature in the range from 35 °C to 120 °C, in particular from 50 °C to 100 °C, and an absolute pressure in the range from 0.1 to 0.9 bar, preferably 0.3 to 0.7 bar, and then the working medium is compressed by the compressor, as a result of which the working medium at the outlet of the compressor has a temperature in the range from 100 °C to 300 °C, preferably from 150 °C to 250 °C, and an absolute pressure in the range from 1 to 10 bar. Then, cooling water is added to the working medium via the mixing element in an amount such that a temperature at the inlet of the reboiler in the range from 80 °C to 200 °C, preferably in the range from 100 °C to 160 °C, more preferably in the range from 110 °C to 160 °C, is established.

The addition of a cooling water to the working medium between the compressor and the downstream reboiler offers the advantage that the temperature of the working medium at the inlet of the reboiler is controlled in a manner that is fast, stable and precise. It is thus possible to deal with elevated temperature peaks before they reach the reboiler through the addition of the cooling water. The allowable temperature range for the polymerizable substances in the reboiler is thus maintained during operation of the distillation plant.

It is preferable when in the method of operating the distillation plant the cooling water is added to the working medium via the mixing element at a temperature in the range from 1 °C to 160 °C, more preferably from 105 °C to 150 °C, in particular from 110 °C to 140 °C, wherein the cooling water has a mass flow rate relative to the mass flow rate of the working medium in the range from 3% to 10%.

Further preferred embodiments of the method of operating the distillation plant correspond to the aforementioned preferred embodiments of the distillation plant according to the invention.

The distillation plant according to the invention is particularly suitable for producing a (meth)acrylate. The invention further provides a process for producing a (meth)acrylate by reacting (meth)acrylic acid with an alcohol corresponding to the (meth)acrylate in the presence of an acidic catalyst and a polymerization inhibitor, comprising the steps of:
providing a rectification column having a reboiler and a vapor condenser, wherein a heat pump circuit comprising a compressor fluidically connects both the reboiler and the vapor condenser and the heat pump circuit employs water as the working medium,
performing an esterification within a reaction zone, wherein the components (meth)acrylic acid and alcohol are employed in a molar ratio in the range from 1.0:1.0 to 1.0:2.0, preferably in the range from 1.0:1.1 to 1.0:1.5, and wherein the esterification is carried out at a temperature in the range from 80 °C to 150 °C, preferably in the range from 100 °C to 130 °C, and at an absolute pressure in the range from 0.2 to 5.0 bar, preferably in the range from 0.4 to 1.5 bar, to obtain a resultant reaction mixture,
separating the water of esterification formed in the esterification from the reaction mixture within the rectification column, wherein the reboiler is heated by the working medium flowing through it in the heat pump circuit,
discharging a gaseous vapor stream enriched by the water of esterification at the top of the rectification column,
condensing the vapor stream in the vapor condenser to form an organic phase and an aqueous phase, wherein the vapor condenser is cooled by the working medium flowing through it in the heat pump circuit and the working medium at the outlet of the vapor condenser has a temperature in the range from 35 °C to 120 °C, in particular from 50 °C to 100 °C, and
supplying the working medium from the outlet of the vapor condenser to the compressor, wherein the working medium is compressed within the compressor and at the outlet of the compressor the working medium has a temperature in the range from 100 °C to 300 °C, preferably from 150 °C to 250 °C, and an absolute pressure in the range from 1 to 10 bar, wherein a mixing element in the heat pump circuit is arranged between the compressor and the downstream reboiler and in order to cool the working medium a cooling water having a temperature in the range from 1 °C to 160 °C, preferably from 105 °C to 150 °C, in particular from 110 °C to 140 °C, is added to the working medium via the mixing element in an amount such that at the inlet of the reboiler the working medium has a temperature in the range from 80 °C to 200 °C, preferably from 100 °C to 160 °C, more preferably from 110 °C to 160 °C.

The addition of a cooling water to the working medium between the compressor and the downstream reboiler has the advantage that the temperature of the working medium at the inlet of the reboiler can be controlled in a manner that is fast, stable and precise. This makes it possible to avoid elevated temperature peaks before they reach the reboiler through the addition of the cooling water. The allowable temperature range for the (meth)acrylate-containing polymerizable substances in the reboiler is thus maintained during operation of the distillation plant.

In the process for producing a (meth)acrylate the working medium cooled down by the reboiler is partially used as cooling water. This technical implementation has the advantage that no further cooling water is required and the heat pump circuit is not disrupted by external cooling water except in exceptional cases. An example of an exceptional case would be if the working medium drains from the heat pump circuit as a result of a leak or if the working medium requires a partial replacement by fresh working medium. Complex safety apparatuses such as pressure valves or complex pressure-attenuating means become redundant as a result. Moreover, it has been found that, surprisingly, the cooling water allows the stipulated temperature range of the working medium to be observed at all times, which means that there is no polymerization, fouling and/or caking on the inner walls of the flow channels of the reboiler.

In the process for producing a (meth)acrylate the cooling water is preferably added at a mass flow rate relative to the mass flow rate of the working medium in the range from 3% to 10%. In a preferred embodiment of the process for producing a (meth)acrylate the cooling water is added at a temperature in the range from 1 °C to 160 °C, more preferably from 105 °C to 150 °C, in particular from 110 °C to 140 °C, and at a mass flow rate relative to the mass flow rate of the working medium in the range from 3% to 10%. This has the advantage that the cooling water is energy-efficiently added to the working medium between the compressor and the downstream reboiler. Only as much cooling water as necessary is added so that the working medium transfers sufficient heat to the reboiler and the temperature of the working medium is not too high.

In the process for producing a (meth)acrylate the working medium partially serves as cooling water, wherein the working medium serving as cooling water is withdrawn from a subregion of the heat pump circuit which extends in the main flow direction of the working medium from the reboiler to the vapor condenser. In particular, a working medium condensate formed in the reboiler is used as cooling water. The working medium serving as cooling water is preferably withdrawn, and/or returned, in particular to the mixing element, at an absolute pressure in a range from 2 to 8 bar, more preferably from 4 to 6 bar. The working medium serving as cooling water is preferably withdrawn, and/or returned, in particular to the mixing element, at a temperature in a range from 105 °C to 150 °C, more preferably from 110 °C to 140 °C.

In a preferred embodiment of the process for producing a (meth)acrylate the reboiler has a downstream condensate vessel in the heat pump circuit and the working medium partially flows from the condensate vessel to the mixing element while serving as cooling water.

In a preferred embodiment of the process for producing a (meth)acrylate the working medium partially flows from the condensate vessel to the compressor while serving as cooling water.

In a preferred embodiment of the process for producing a (meth)acrylate the compressor comprises one compression stage or two or more compression stages and in the case of two or more compression stages the cooling water is at least partially added to the working medium via a respective intermediate-stage mixing element per compression stage, wherein the respective intermediate-stage mixing element is arranged between the respectively adjacent compression stages of the compressor.

In a preferred embodiment of the process for producing a (meth)acrylate a respective liquid droplet separator is connected downstream of each intermediate-stage mixing element between the respectively adjacent compression stages.

In a preferred embodiment of the process for producing a (meth)acrylate in order to cool the working medium a cooling water having a temperature in the range from 1 °C to 160 °C, more preferably from 105 °C to 150 °C, in particular from 110 °C to 140 °C, is added to the working medium via the intermediate-stage mixing element in an amount such that at the inlet of the downstream compression stage a temperature difference between the temperature of the working medium and the temperature at which under the prevailing absolute pressure the working medium is in the form of saturated steam in the range from 2 °C to 50 °C, preferably in the range from 5 °C to 20 °C, is established. In this embodiment of the process the cooling water is preferably added via the intermediate-stage mixing element at a mass flow rate relative to the mass flow rate of the working medium at the inlet of the compressor in the range from 3% to 10%. This has the advantage that the cooling water is energy-efficiently added to the working medium between the compression stages so that only as much cooling water as necessary is added.

In a preferred embodiment of the process for producing a (meth)acrylate the employed mixing element and/or the intermediate-stage mixing element is a mixing nozzle, a steam jet or a venturi pump.

In a preferred embodiment of the process for producing a (meth)acrylate a phase separator is connected downstream of the vapor condenser, wherein both an aqueous phase and an organic phase are formed in the phase separator and these two phases are at least partially recycled to the rectification column, wherein the two phases are preferably recycled separately to the rectification column through two separate recycle conduits.

In a preferred embodiment of the process for producing a (meth)acrylate a further vapor condenser downstream of the vapor condenser is present and this further vapor condenser comprises a downstream phase separator, wherein both an aqueous phase and an organic phase are formed in the phase separator. It is preferable when one of these two phases is at least partially recycled to the rectification column. It is very particularly preferable when both phases are each separately at least partially recycled to the rectification column through two separate recycle conduits.

In a preferred embodiment of the process for producing a (meth)acrylate the walls of the reboiler, across which the bottoms mixture from the rectification column flows during operation of the distillation plant, are made from a nonrusting and acid-resistant material selected from the group consisting of zirconium, stainless steels and nickel-based alloys.

In a preferred embodiment of the process for producing a (meth)acrylate heat pump circuit comprises a water separator, wherein the working medium flows from the water separator into the vapor condenser and then the working medium flows back from the vapor condenser into the water separator.

In a preferred embodiment of the process for producing a (meth)acrylate in order to cool the working medium a cooling water having a temperature in the range from 1 °C to 160 °C, more preferably from 105 °C to 150 °C, in particular from 110 °C to 140 °C, is added to the working medium via the mixing element in an amount such that at the inlet of the reboiler a temperature difference between the temperature of the working medium and the temperature at which under the prevailing absolute pressure the working medium is in the form of saturated steam in the range from 5 °C to 50 °C, more preferably from 5 °C to °C, can be established or is established, wherein the prevailing absolute pressure is preferably in the range from 2 to 8 bar, particularly preferably in the range from 4 to 6 bar. In particular in this preferred embodiment the cooling water is preferably added via the mixing element at a mass flow rate relative to the mass flow rate of the working medium in the range from 3% to 10%.

In a particularly preferred embodiment of the process for producing a (meth)acrylate the (meth)acrylate is an n-butyl (meth)acrylate.

The particularly preferred process for producing an n-butyl (meth)acrylate is based on the reactants n-butanol and (meth)acrylic acid. In the present document “(meth)acrylic acid” is used to refer to a (meth)acrylic acid quality which preferably comprises at least 98% by weight, more preferably at least 99.5% by weight, of (meth)acrylic acid, and in addition preferably not more than 0.2% by weight of water and also preferably not more than 0.03% by weight in each case of acetic acid, propionic acid and isobutyric acid. It is preferable to employ an n-butanol quality comprising at least 99.5% by weight of n-butanol, not more than 0.05% n-butanal, not more than 0.02% dibutyl ether, not more than 0.1% other alcohols and not more than 0.05% water. The color number is preferably not more than APHA 5, the acid number not more than 0.03 mgKOH/g.

Suitable polymerization inhibitors in the production of (meth)acrylate, which act as stabilizers, include for example N-oxides (nitroxyl or N-oxyl radicals, i.e. compounds having at least one NO group) such as 4-hydroxy-2,2,6,6-tetramethylpiperidine-N-oxyl (HO-TEMPO), 4-oxo-2,2,6,6-tetramethylpiperidine-N-oxyl, 4-acetoxy-2,2,6,6-tetramethylpiperidine-N-oxyl, 2,2,6,6-tetramethylpiperidine-N-oxyl, bis(1-oxyl-2,2,6,6-tetramethylpiperidin-4-yl) sebacate, 4,4′,4″-tris (2,2,6,6-tetramethylpiperidine-N-oxyl) phosphite or 3-oxo-2,2,5,5-tetramethylpyrrolidine-N-oxyl; monohydric or polyhydric phenols optionally having one or more alkyl groups, such as alkylphenols, for example o-, m- or p-cresol (methylphenol), 2-tert-butylphenol, 4-tert-butylphenol, 2,4-di-tert-butylphenol, 2-methyl-4-tert-butylphenol, 2-tert-butyl-4-methylphenol, 2,6-tert-butyl-4-methylphenol, 4-tert-butyl-2,6-dimethylphenol or 6-tert-butyl-2,4-dimethylphenol; quinones such as hydroquinone, hydroquinone monomethyl ether, 2-methylhydroquinone or 2,5-di-tert-butylhydroquinone; hydroxyphenols such as catechol (1,2-dihydroxybenzene) or benzoquinone; aminophenols such as p-aminophenol; nitrosophenols such as p-nitrosophenol; alkoxyphenols such as 2-methoxyphenol (guaiacol, catechol monomethyl ether), 2-ethoxyphenol, 2-isopropoxyphenol, 4-methoxyphenol (hydroquinone monomethyl ether), mono- or di-tert-butyl-4-methoxyphenol; tocopherols such as a-tocopherol and 2,3-dihydro-2,2-dimethyl-7-hydroxybenzofuran (2,2-dimethyl-7-hydroxycoumaran), aromatic amines such as N,N-diphenylamine or N-nitrosodiphenylamine; phenylenediamines such as N,N′-dialkyl-p-phenylenediamine, where the alkyl radicals may be the same or different and each independently consist of 1 to 4 carbon atoms and may be straight-chain or branched, for example N,N′-dimethyl-p-phenylenediamine or N,N′-diethyl-p-phenylenediamine, hydroxylamines such as N, N-diethylhydroxylamine, imines such as methylethylimine or methylene violet, sulfonamides such as N-methyl-4-toluenesulfonamide or N-tert-butyl-4-toluenesulfonamide, oximes such as aldoximes, ketoximes or amidoximes such as diethyl ketoxime, methyl ethyl ketoxime or salicylaldoxime, phosphorus-containing compounds such as triphenylphosphine, triphenyl phosphite, triethyl phosphite, hypophosphorous acid or alkyl esters of phosphorous acids; sulfur-containing compounds such as diphenyl sulfide or phenothiazine; metal salts such as copper or manganese, cerium, nickel, and chromium salts, for example chlorides, sulfates, salicylates, tosylates, acrylates or acetates, for example copper acetate, copper (II) chloride, copper salicylate, cerium (III) acetate or cerium (III) ethylhexanoate, or mixtures thereof.

The polymerization inhibitor or polymerization inhibitor mixture used in the production of (meth)acrylate is preferably at least one compound from the group of hydroquinone, hydroquinone monomethyl ether, phenothiazine, 4-hydroxy-2,2,6,6-tetramethylpiperidine-N-oxyl, 4-oxo-2,2,6,6-tetramethylpiperidine-N-oxyl, bis(1-oxyl-2,2,6,6-tetramethylpiperidin-4-yl) sebacate, 2-tert-butylphenol, 4-tert-butylphenol, 2,4-di-tert-butylphenol, 2-tert-butyl-4-methylphenol, 6-tert-butyl-2,4-dimethylphenol, 2,6-di-tert-butyl-4-methylphenol, 2-methyl-4-tert-butylphenol, hypophosphorous acid, copper (II) acetate, copper (I) chloride, copper (II) chloride, copper (II) salicylate and cerium (III) acetate.

Particular preference is given to using phenothiazine (PTZ) and/or hydroquinone monomethyl ether (MEHQ) and/or HO-Tempo as polymerization inhibitor in the production of n-butyl (meth)acrylate.

In the production of n-butyl (meth)acrylate the polymerization inhibitor is preferably dissolved in one or more liquid organic compounds. The organic compound is preferably 2-butanol and/or n-butyl (meth)acrylate in the production of n-butyl (meth)acrylate.

Suitable esterification catalysts in the production of (meth)acrylate include the standard mineral acids and sulfonic acids, preferably sulfuric acid, phosphoric acid, alkylsulfonic acids (e.g. methanesulfonic acid, trifluoromethanesulfonic acid) and arylsulfonic acids (e.g. benzenesulfonic acid, p-toluenesulfonic acid or dodecylbenzenesulfonic acid) or mixtures thereof, but acidic ion exchangers or zeolites are also usable.

In the production of (meth)acrylate it is particularly preferable to employ sulfuric acid, methanesulfonic acid, p-toluenesulfonic acid, m-toluenesulfonic acid, o-toluenesulfonic acid or mixtures thereof.

It is very particularly preferable to employ p-toluenesulfonic acid as esterification catalyst in the production of n-butyl (meth)acrylate. In the reaction zone, which is preferably within the reactor, the content thereof based on the reaction mixture present therein is advantageously 0.1-10.0% by weight, preferably 0.1-6.0% by weight. Other organic sulfonic acids such as methanesulfonic acid, benzenesulfonic acid, dodecylbenzenesulfonic acid and/or sulfuric acid are likewise usable. The amount thereof is equimolar to that of para-toluenesulfonic acid. Corresponding mixtures are also possible. The content of catalytically active acid in the bottom of the rectification column based on the mixture present therein may advantageously be between 2.5% and 50.0% by weight of para-toluenesulfonic acid or an equimolar amount of another organic sulfonic acid and/or sulfuric acid.

The invention shall now be more particularly elucidated with reference to the drawing. The drawing is to be considered as schematic diagrams. It does not represent a limitation of the invention, for example with regard to specific dimensions or design variants. The drawing shows:

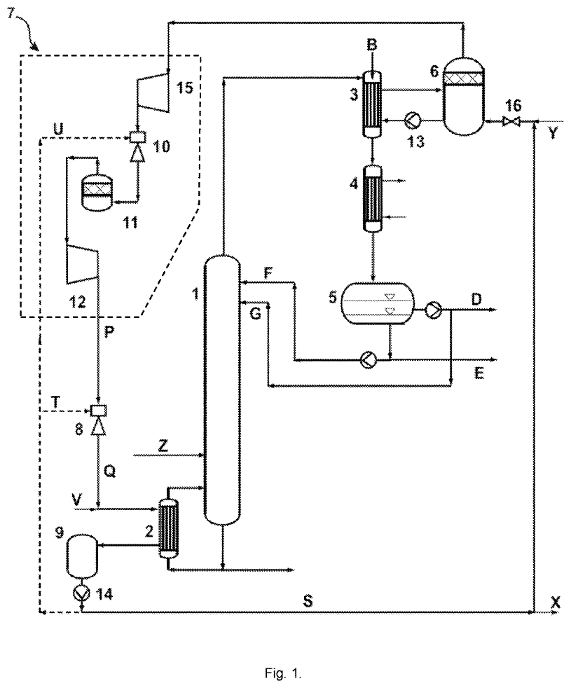

FIG. 1: A schematic process flow diagram of an illustrative distillation plant of the invention for production of a (meth)acrylate using a heat pump.

### LIST OF REFERENCE NUMBERS USED

B conduit for supplying the polymerization inhibitor
D conduit for discharging an organic phase from a phase separator
E conduit for discharging an aqueous phase from a phase separator
F conduit for supplying an aqueous phase from a phase separator to a rectification column
G conduit for supplying an organic phase from a phase separator to a rectification column
P conduit for supplying working medium from a compressor to a mixing element
Q conduit for supplying working medium from a mixing element to a reboiler
S conduit for supplying working medium from a condensate vessel to a water separator
T conduit for supplying working medium from a condensate vessel to a mixing element
U conduit for supplying working medium from a condensate vessel to an intermediate-stage mixing element
V conduit for supplying external working medium
X conduit for discharging working medium from the heat pump circuit
Y conduit for supplying external working medium
Z conduit for supplying a mixture resulting from a reactor
1 rectification column
2 reboiler
3 first vapor condenser
4 second vapor condenser
5 phase separator
6 water separator
7 compressor
8 mixing element
9 condensate vessel
10 intermediate-stage mixing element between two compression stages
11 liquid droplet separator between two compression stages
12 second compression stage of a compressor
13 pump for conveying the working medium from the water separator to the vapor condenser
14 pump for conveying the working medium from the condensate vessel to the water separator
15 first compression stage of the compressor
16 expansion valve

FIG. 1 shows a schematic of a process flow diagram of an illustrative process of the invention for producing polymerizable substances, preferably for producing a (meth)acrylate, in which a mixture resulting from a reaction zone is conveyed into a rectification column 1 via a conduit Z.

At the top of the rectification column 1, a vapor stream is discharged. In a downstream vapor condenser 3, the vapor stream is partly condensed to form an organic phase and an aqueous phase. The vapor condenser 3 here is an upright shell-and-tube heat exchanger 3, wherein one or more polymerization inhibitors present in a solution are added via a conduit B in the inlet region of the shell-and-tube heat exchanger 3. Moreover, the shell-and-tube heat exchanger 3 is equipped with spray nozzles connected to the conduit B. The spray nozzles distribute the polymerization inhibitor(s) present in the solution onto the inside of the tubes of the shell-and-tube heat exchanger 3. This directly stabilizes the vapor stream on condensate formation, which efficiently prevents polymer formation.

A further vapor condenser 4 downstream of the vapor condenser 3 condenses the low boilers remaining in the vapor stream, which results in complete condensation of the vapor stream in the vapor condenser 4.

Subsequently, the condensate is fed to a phase separator 5. The organic phase is returned to the rectification column 1 at least partly via a conduit G, with the remaining proportion of the organic phase being discharged from the distillation plant via a conduit D. A separate conduit F is used to return the aqueous phase at least partly to the rectification column 1, with the remaining proportion of the aqueous phase being discharged from the distillation plant via a conduit E.

A reboiler 2 heats the bottoms from the rectification column 1 in that at least a portion of the bottoms output flows through the reboiler 2 and is then returned back to the column bottom. The remaining proportion of the bottoms output is discharged from the distillation plant.

The first vapor condenser 3 is cooled by the working medium that flows through it in the heat pump circuit. The working medium flows from the outlet of the vapor condenser 3 through a water separator 6 to a compressor 7, wherein the water separator 6 includes a demister.

The compressor 7 comprises a first compression stage 15 and a second compression stage 12. The compressor 7 compresses the working medium. The working medium then flows via a conduit P from the outlet of the compressor 7 into a mixing element 8. The working medium then flows from the mixing element 8 via a conduit Q to the inlet of the reboiler 2. Subsequently, the working medium flows from the outlet of the reboiler 2 into a condensate vessel 9. A pump 14 conveys a substream of the working medium that serves as cooling water to the mixing element 8 in a conduit T. The mixing element 8 then adds the working medium that serves as cooling water to the working medium that flows in from the compressor 7.

By means of the pump 14, a conduit U is used to convey a further substream of the working medium that serves as cooling water from the condensate vessel 9 to the intermediate-stage mixing element 10. A liquid droplet separator 11 which is connected downstream of the intermediate-stage mixing element 10, and which preferably comprises a knitted wire mesh, protects the second compression stage 12 from liquid droplets.

By means of the pump 14, a further substream of the working medium is conveyed from the outlet of the condensate vessel 9 to the inlet of the water separator 6 via a conduit S, where the working medium is expanded by means of an expansion valve 16 prior to entry or preferably on entry into the water separator 6. Subsequently, the working medium is conveyed by a pump 13 from the water separator 6 to the vapor condenser 3.

In an exceptional case, by means of a conduit V, it is also possible to add external steam, serving as working medium, to the conduit Q, in order to be able to increase the temperature of the working medium at the inlet of the reboiler 2. In an exceptional case, by means of a conduit Y, it is also possible to add external water, serving as working medium, to the water separator 6.

The working medium may be removed at any time from the heat pump circuit via a conduit X if, for example, too great a pressure builds up in the heat pump circuit or the working medium is to be exchanged.

### EXAMPLES

The examples of the process for producing n-butyl acrylate that follow are simulated by thermodynamic simulations. For this purpose, the Aspen Plus® software (Aspen) was used, which can be found at the website https://www.aspentech.com. Aspen is an extensive simulation software package which is used for modeling, simulation and optimization of chemical processes and plants in industry. Aspen has extensive model databases for modeling of basic operations and substance databases for the physical properties of many different substances.

### Example 1

A thermodynamic simulation of an inventive embodiment of the process for producing n-butyl acrylate using a distillation plant according to FIG. 1 was conducted in Aspen and gives the following results:

A mixture resulting from a reactor is fed via a conduit Z to a rectification column 1 at a mass flow rate of 30 010 kg/h and at a temperature of 105 °C, where the mixture has the following composition in parts by weight:

| | |
|---|---|
| Butanol | 0.079 |
| n-butyl acrylate | 0.650 |
| acrylic acid | 0.070 |
| water | 0.003 |
| n-butyl n-butoxypropionate | 0.098 |
| balance | 0.100 |

At the top of the rectification column 1, a vapor stream is discharged at a temperature of 88 °C. The mass flow rate of the vapor stream is 37 350 kg/h and the vapor stream has the following composition in parts by weight:

| | |
|---|---|
| water | 0.381 |
| butanol | 0.086 |
| n-butyl acrylate | 0.526 |
| balance | 0.007 |

Subsequently, the vapor stream is condensed in an upright shell-and-tube heat exchanger as vapor condenser 3 and in a further, downstream upright shell-and-tube heat exchanger as further vapor condenser 4 to form an organic phase and an aqueous phase. The condensate has a temperature of 32 °C and an absolute pressure of 0.8 bar. The vapor condenser 3 is equipped with spray nozzles in the inlet region that distribute a solution comprising one or more polymerization inhibitors onto the inside of the tubes of the shell-and-tube heat exchanger. The mass flow rate of the solution here is 90 kg/h. On formation of the condensate, this stabilizes the condensate directly and consequently prevents polymer formation.

Subsequently, the condensate is fed to a phase separator 5. The organic phase that forms is returned to the rectification column 1 via a conduit G at a mass flow rate of 8062 kg/h and discharged from the distillation plant via a conduit D at a mass flow rate of 15 250 kg/h. The aqueous phase that forms is returned to the rectification column 1 via a conduit F at a mass flow rate of 15 590 kg/h and separately discharged from the distillation plant via a conduit E at a mass flow rate of 1855 kg/h.

A bottoms outflow with a mass flow rate of 377 400 kg/h and at a temperature of 95 °C from the bottom of the rectification column 1 is heated to a temperature of 95 °C by a reboiler 2 and returned to the rectification column 1. A further bottoms outflow with a mass flow rate of 16 330 kg/h and at a temperature of 95 °C is discharged from the distillation plant.

The mixture in the bottom of the rectification column 1 has the following composition here in parts by weight:

| | |
|---|---|
| water | 0.356 |
| acrylic acid | 0.130 |
| n-butyl n-butoxypropionate | 0.195 |
| acryloyloxy ester | 0.096 |
| high boilers | 0.098 |
| balance | 0.147 |

The vapor condenser 3 is cooled by the working medium that flows through it in the heat pump circuit. The mass flow rate of the working medium here is 78 250 kg/h, and the working medium at the outlet of the vapor condenser 3 is at a temperature of 80 °C and an absolute pressure of 0.47 bar. In the simulation, the working medium used is water.

The working medium flows at a mass flow rate of 17 250 kg/h from the outlet of the vapor condenser 3 via a liquid droplet separator 6 to a compressor 7.

At the inlet of the compressor 7, the working medium has a temperature of 80 °C and an absolute pressure of 0.47 bar. The compressor 7 comprises two compression stages 15, 12 and is a two-stage geared turbocompressor. The compressor 7 compresses the working medium such that the working medium has an absolute pressure of 2.8 bar and a temperature of 206 °C at the outlet of the compressor 7.

The working medium then flows from the outlet of the compressor 7 through a conduit P to a steam jet nozzle that functions as mixing element 8. Subsequently, the working medium flows through a conduit Q from the steam jet nozzle to the inlet of the reboiler 2. By virtue of the intermediately inserted steam jet nozzle, a working medium that serves as cooling water is added to the working medium, so that the working medium at the inlet of the reboiler 2, configured as a shell-and-tube evaporator, has a temperature of 140 °C, an absolute pressure of 2.8 bar and a mass flow rate of 19 980 kg/h. This is achieved in that a pump 14 conveys a substream of the working medium, serving as cooling water, from the outlet of the condensate vessel 9 at a mass flow rate of 1085 kg/h and at a temperature of 131 °C via a conduit T to the steam jet nozzle. The steam jet nozzle then adds the working medium that serves as cooling water to the working medium that flows in from the compressor 7.

By means of the pump 14, a further substream of the working medium is conveyed at a mass flow rate of 1644 kg/h and at a temperature of 131 °C via a conduit U from the outlet of the condensate vessel 9 to a further steam jet nozzle, where the further steam jet nozzle functions as an intermediate-stage mixing element 10 and is present between the first compression stage 15 and the second compression stage 12. The further steam jet nozzle adds the working medium serving as cooling water to the working medium flowing in from the first compression stage 15. A liquid droplet separator 11 downstream of the further steam jet nozzle protects the downstream compression stage 12 from liquid droplets. The temperature difference between the temperature of the working medium at the inlet into the downstream compression stage 12 and the saturated steam temperature of the working medium at the existing pressure is 10 °C.

A further substream of the working medium is conveyed by the pump 14 at a mass flow rate of 17 250 kg/h and at a temperature of 131 °C through a conduit S from the outlet of the condensation vessel 9 to the inlet of the liquid droplet separator 6, where the working medium is expanded by means of an expansion valve 16 before entry into the droplet separator 6. Immediately before the working medium flows through the expansion valve 16, the temperature of the working medium is 131 °C and the absolute pressure of the working medium is 4 bar.

Subsequently, a pump 13 conveys the working medium at a temperature of 80 °C and at an absolute pressure of 1 bar from the liquid droplet separator 6 to the vapor condenser 3.

### Comparative Example 1

With the exception of the features specified hereinafter, this comparative example 1 is set up in the same way as example 1. Here too, a thermodynamic simulation was conducted with the Aspen software.

The working medium is not cooled by a mixing element 8 after exiting from the compressor 7. As a result, the working medium at the inlet of the reboiler 2 is at a temperature of 260 °C, rather than the temperature of 140 °C that occurs in example 1. The mass flow rate of the working medium in the conduit P in this comparative example is 18 890 kg/h, rather than the mass flow rate of 19 980 kg/h in example 1. The higher temperature of the working medium also gives rise to higher temperatures in the reboiler 2. As a result of the dynamic characteristics of the distillation plant, higher temperature peaks thus also occur, which excessively heat the bottoms mixture flowing through the reboiler 2. In practical terms, this can result in fouling and/or polymer formation in the reboiler 2. In practical terms, the plant would then have to be shut down at shorter intervals for cleaning.

### Comparative Example 2

With the exception of the features specified, this comparative example 2 is set up in the same way as example 1. Here too, a thermodynamic simulation was conducted with the Aspen software.

By comparison with example 1, no working medium that serves as cooling water is added to the mixing element 8, nor to the intermediate-stage mixing element 10. Instead, in this comparative example 2, the working medium is cooled down by appropriate heat exchangers at these two points. The use of the heat exchangers reduces both the mass flow rate of the working medium exiting from the compressor 7 from 18 890 kg/h to 17 250 kg/h and the mass flow rate of the working medium entering the reboiler 2 from 19 980 kg/h to 17 250 kg/h.

Under these conditions, a total of 1569 KW of heat is withdrawn from the heat pump circuit by the two heat exchangers. Owing to the reduced flow of working medium through the reboiler 2, less heat is transferred to the bottoms mixture flowing through the reboiler 2. Instead, an external heating medium, for example a heating steam, has to be added to the reboiler 2. This also reduces the efficiency of the heat pump circuit.

1.-16. (canceled)

17. A distillation plant for polymerizable substances comprising a rectification column having a reboiler and a vapor condenser, wherein a heat pump circuit comprising a compressor fluidically connects both the reboiler and the vapor condenser and is configured such that during operation of the distillation plant the reboiler is heated by the heat pump circuit and the vapor condenser is cooled by the heat pump circuit, wherein the heat pump circuit employs water as the working medium, wherein
a mixing element is arranged between the compressor and the downstream reboiler, wherein the mixing element is configured such that during operation of the distillation plant in order to cool the working medium a cooling water is added in an amount such that a temperature of the working medium in the range from 80 °C to 200 °C can be established at the inlet of the reboiler and wherein the distillation plant is configured such that during operation of the distillation plant the working medium partially serves as cooling water, wherein the working medium serving as cooling water is withdrawn from a subregion of the heat pump circuit which extends in the main flow direction of the working medium from the reboiler to the vapor condenser.

18. The distillation plant according to claim 17, wherein the reboiler has a downstream condensate vessel in the heat pump circuit and both the condensate vessel and the mixing elements are adapted such that during operation of the distillation plant the working medium partially flows from the condensate vessel to the mixing element while serving as cooling water.

19. The distillation plant according to claim 18, wherein both the condensate vessel and the compressor are adapted such that during operation of the distillation plant the working medium partially flows from the condensate vessel to the compressor while serving as cooling water.

20. The distillation plant according to claim 17, wherein the compressor comprises one compression stage or two or more compression stages and the distillation plant is adapted such that during operation of the distillation plant the cooling water in the case of two or more compression stages is at least partially added to the working medium via a respective intermediate-stage mixing element per compression stage, wherein the respective intermediate-stage mixing element is arranged between the respectively adjacent compression stages of the compressor.

21. The distillation plant according to claim 20, wherein in the case of two or more compression stages a respective liquid droplet separator is connected downstream of each intermediate-stage mixing element between the respectively adjacent compression stages.

22. The distillation plant according to claim 17, wherein the mixing element and/or the respective intermediate-stage mixing element present in the case of two or more compression stages is a mixing nozzle, a steam jet or a venturi pump.

23. The distillation plant according to claim 17, wherein a phase separator is connected downstream of the vapor condenser, wherein the phase separator is configured such that during operation of the distillation plant both an aqueous phase and an organic phase are formed in the phase separator and these two phases are at least partially recycled to the rectification column, wherein the two phases are preferably separately recycled to the rectification column via two separate recycle conduits.

24. The distillation plant according to claim 17, wherein the heat pump circuit comprises a water separator, wherein the water separator is fluidically connected to the vapor condenser in such a way that during operation of the distillation plant the working medium flows from the water separator into the vapor condenser and then the working medium flows back from the vapor condenser into the water separator.

25. A process for producing a (meth)acrylate by reacting (meth)acrylic acid with an alcohol corresponding to the (meth)acrylate in the presence of an acidic catalyst and a polymerization inhibitor, comprising the steps of:
providing a rectification column having a reboiler and a vapor condenser, wherein a heat pump circuit comprising a compressor fluidically connects both the reboiler and the vapor condenser and the heat pump circuit employs water as the working medium,
performing an esterification within a reaction zone, wherein the components (meth)acrylic acid and alcohol are employed in a molar ratio in the range from 1.0:1.0 to 1.0:2.0, and wherein the esterification is carried out at a temperature in the range from 80 °C to 150 °C, and at an absolute pressure in the range from 0.2 to 5.0 bar, to obtain a resultant reaction mixture,
separating the water of esterification formed in the esterification from the reaction mixture within the rectification column, wherein the reboiler is heated by the working medium flowing through it in the heat pump circuit,
discharging a gaseous vapor stream enriched by the water of esterification at the top of the rectification column,
condensing the vapor stream in the vapor condenser to form an organic phase and an aqueous phase, wherein the vapor condenser is cooled by the working medium flowing through it in the heat pump circuit and the working medium at the outlet of the vapor condenser has a temperature in the range from 35 °C to 120 °C and
supplying the working medium from the outlet of the vapor condenser to the compressor, wherein the working medium is compressed within the compressor and at the outlet of the compressor the working medium has a temperature in the range from 100 °C to 300 °C and an absolute pressure in the range from 1 to 10 bar, wherein a mixing element in the heat pump circuit is arranged between the compressor and the downstream reboiler, wherein in order to cool the working medium a cooling water having a temperature in the range from 1 °C to 160 °C is added to the working medium via the mixing element in an amount such that at the inlet of the reboiler the working medium has a temperature in the range from 80 °C to 200 °C and
the working medium partially serves as cooling water, wherein the working medium serving as cooling water is withdrawn from a subregion of the heat pump circuit which extends in the main flow direction of the working medium from the reboiler to the vapor condenser.

26. The process according to claim 25, wherein the cooling water is added at a temperature in the range from 1 °C to 160 °C and at a mass flow rate relative to the mass flow rate of the working medium in the range from 3% to 10%.

27. The process according to claim 25, wherein the compressor comprises one compression stage or two or more compression stages and in the case of two or more compression stages the cooling water is at least partially added to the working medium via a respective intermediate-stage mixing element per compression stage, wherein the respective intermediate-stage mixing element is arranged between the respectively adjacent compression stages of the compressor.

28. The process according to claim 27, wherein in order to cool the working medium the cooling water having a temperature in the range from 1 °C to 160 °C is added to the working medium via the intermediate-stage mixing element in an amount such that at the inlet of the downstream compression stage a temperature difference between the temperature of the working medium and the temperature at which under the prevailing absolute pressure the working medium is in the form of saturated steam in the range from 2 °C to 50 °C is established.

29. The process according to claim 25, wherein in order to cool the working medium the cooling water having a temperature in the range from 1 °C to 160 °C is added to the working medium via the mixing element in an amount such that at the inlet of the reboiler a temperature difference between the temperature of the working medium and the temperature at which under the prevailing absolute pressure the working medium is in the form of saturated steam in the range from 5 °C to 50 °C can be established.

30. The process according to claim 25, wherein the (meth)acrylate is an n-butyl (meth)acrylate.

31. The use of a distillation plant according to claim 17, wherein the distillation plant is employed in chemical processes.

32. A method of operating a distillation plant according to claim 17, wherein a liquid mixture is conveyed into the rectification column and
during the distillative separation a vapor stream having a temperature in the range from 35 °C to 120 °C is formed at the inlet of the vapor condenser and a bottoms product having a temperature in the range from 80 °C to 160 °C is formed in the bottom of the rectification column and the working medium from the reboiler is at least partially supplied to the vapor condenser to be cooled and the working medium is heated in the vapor condenser, wherein the working medium at the outlet of the vapor condenser has a temperature in the range from 35 °C to 120 °C and an absolute pressure in the range from 0.1 to 0.9 bar, and
then the working medium is compressed by the compressor, as a result of which the working medium at the outlet of the compressor has a temperature in the range from 100 °C to 300 °C and an absolute pressure in the range from 1 to 10 bar, and
then cooling water is added to the working medium via the mixing element in an amount such that a temperature at the inlet of the reboiler in the range from 80 °C to 200 °C is established.
